(19) United States
(12) Patent Application Publication
(10) Pub. No.: US 2024/0177856 A1
(43) Pub. Date: May 30, 2024

(54) CLINICAL DECISION SUPPORT SYSTEMS EMPLOYING REVERSE PHENOTYPING

(71) Applicant: Albert Einstein College of Medicine, Bronx, NY (US)

(72) Inventor: John Greally, New York, NY (US)

(21) Appl. No.: 18/282,671

(22) PCT Filed: Mar. 16, 2022
(86) PCT No.: PCT/US22/20590

§ 371 Date: Sep. 18, 2023

Related U.S. Application Data
(60) Provisional application No. 63/161,660, filed on Mar. 16, 2021.

(51) Int. Cl. G16H 50/20; G16B 20/20; G16B 40/20; G16H 10/60

(52) U.S. Cl. CPC G16H 50/20; G16B 20/20; G16B 40/20; G16H 10/60

(57) ABSTRACT

Clinical decision support systems employing reverse phenotyping. In various embodiments, genomic information of a patient is read from a datastore. One or more variant of the genomic information is determined. The one or more variant is associated with a disease state. One or more phenotypic features are determined, associated with the one or more variant. A healthcare provider is prompted to evaluate the patient for the one or more phenotypic features.

### CROSS-REFERENCE TO RELATED APPLICATIONS

This application claims the benefit of U.S. Provisional Application No. 63/161,660, filed Mar. 16, 2021, which is hereby incorporated by reference in its entirety.

### BACKGROUND

Embodiments of the present disclosure relate to systems for clinical decision support, and more specifically, to clinical decision support systems employing reverse phenotyping.

### BRIEF SUMMARY

According to embodiments of the present disclosure, methods of and computer program products for clinical decision support are provided. Genomic information of a patient is read from a datastore. One or more variant of the genomic information is determined. The one or more variant is associated with a disease state. One or more phenotypic features are determined, associated with the one or more variant. A healthcare provider is prompted to evaluate the patient for the one or more phenotypic features.

In some embodiments, reading the genomic information of the patient comprises accessing an electronic health record of the patient. In some embodiments, reading the genomic information of the patient comprises accessing a sequencing provider.

In some embodiments, determining the one or more variant comprises comparing the genomic information of the patient to a reference sequence. In some embodiments, determining the one or more variant comprises accessing a datastore containing associations between variants and disease states.

In some embodiments, determining the one or more phenotypic features comprises accessing a datastore containing associations between variants and phenotypes.

In some embodiments, determining the one or more phenotypic features comprises providing the one or more variant to a trained learning systems, and obtaining therefrom the one or more phenotypic features.

In some embodiments, prompting the healthcare provider comprises displaying the one or more phenotypic features in an electronic health record interface.

In some embodiments, an evaluation of the one or more phenotypic features in the patient is received from the healthcare provider. In some embodiments, the one or more variant and the evaluation of the one or more phenotypic features is provided to a learning system, thereby training the learning system to associate the one or more variant and the one or more phenotypic features. In some embodiments, the phenotypic features of the patient are stored in an electronic health record of the patient.

### BRIEF DESCRIPTION OF THE SEVERAL VIEWS OF THE DRAWINGS

FIG. 1 is a schematic diagram of a system for medical decision support employing reverse phenotyping according to embodiments of the present disclosure.

FIG. 2 is a schematic diagram illustrating reverse phenotyping in comparison to alternative genomic diagnostic approaches.

FIG. 3 illustrates a method of clinical decision support according to embodiments of the present disclosure.

FIG. 4 depicts a computing node according to an embodiment of the present disclosure.

### DETAILED DESCRIPTION

Clinical Decision Support (CDS) systems may be used for many types of information in medicine. In essence, these systems take available data and use them to prompt the clinician to make optimal management decisions. In the field of genomics, CDS may be used for two exemplary applications—pharmacogenomics and cancer genomics. Pharmacogenomics CDS uses variant information to flag potential adverse drug reactions for several drugs. Cancer genomics looks for mutations that prompt the use of specific medications in cancer therapies, or uses genomic information to predict the severity of disease and the need for more robust therapy in high-risk individuals. Other genomic CDS applications are possible, such as polygenic risk scores for risk stratification for common diseases.

Reverse phenotyping may be used in a research context to explore genetic variation. However, there remains a need for effective use of reverse phenotyping in the context of clinical care. Clinical decision support (CDS) using genomics data may be used in personalized medicine. Such applications include focus on medically actionable genetic variants, such as those in cancer genomes that help guide targeted biological therapies. For example, genomic predictors of common diseases may be determined through polygenic risk scores.

In various embodiments of the disclosure, a software system is provided that uses genomic information to prompt a clinician to look for specific features in a patient that could be components of a disease caused by a pathogenic variant in one of their genes. This marks a departure from general diagnostic procedures, in which phenotypic features in the patient may be identified by a clinician, who tries to make sense of them as a group occurring in a known disease and then orders follow-up tests.

Various embodiments of the present disclosure use genome-wide, (exome or whole genome) sequencing performed on the patient. However, it will be appreciated that while WGS may quickly become the dominant source of individual genomics data, the present disclosure is also applicable to exome data, or SNP microarray data.

It will be appreciated that the ability to generate phenotypic information depends on the extant knowledge about the phenotypic features associated with each gene. Both the number of annotated genes and the number of phenotypic terms per gene are rapidly increasing. Accordingly, systems as set forth herein are particularly useful now and in the immediate future.

Using patient sequencing data, a potential disease diagnosis not previously suspected to be occurring in the patient is prompted by their genomic data. This is beneficial for the health care of the patient in terms of quality, cost, and outcomes of care. As reference databases grow to include phenotypic features for each gene known to cause disease, more extensive prompts may be provided to a clinician to look specifically for those features. Reverse phenotyping may be used to describe the use of genomic sequence data in this way to prompt the focused assessment of the patient to see whether they have any identifiable manifestations of a genetic disease.

Medical genomics decision support systems based on reverse phenotyping permit the widespread use of genomics in patient care. In various embodiments, a provider-facing software interface is integrated with an electronic health record system that prompts the clinician to look for specific phenotypic features in a patient for whom genomic information is available. Phenotypic features may be ascertained from physical examination (e.g., dysmorphic features), blood tests (e.g., hyperlipidemia), imaging (e.g., abdominal ultrasound) or other testing (e.g., electrocardiography). If a feature is found that indicates a sequence variant is causing disease, this will prompt a formal request for an analysis of that specific variant and gene by the clinical genetics diagnostic laboratory, which can then be used in patient management.

In various embodiments, diagnostic re-analysis may be performed when the initial testing is negative. For example, systems according to the present disclosure tool may be used to facilitate diagnostic re-analysis by prompting a clinician to categorize certain phenotypic terms as present, absent, or status not known in a patient when reanalyzing a patient for whom prior genomic testing was negative. These phenotypic terms can be derived from multiple sources including genomic information. This use case may be limited to situations where a given clinical indication is already provided.

Reverse phenotyping-based medical genomics decision support systems according to the present disclosure help democratize the use of genomics in clinical care. For example, medical genomics data is often provided in an official report of a diagnostic laboratory, presented in a complex manner that mostly requires specialist medical geneticist interpretation. In this approach, input into the diagnostic process by medical geneticists or clinicians from other specialties is minimal. Furthermore, when performing genome-wide testing, reporting may be limited to a limited panel of medically-actionable genes, while the information from the remainder of the genome is not available for use in the care of the patient. The present disclosure addresses this problem through a medical genomics decision support system with reverse phenotyping. This enables clinicians, even those not trained in genetics, to use complex genomic sequence data effectively and responsibly, with benefits for the patient in terms of their care.

In an exemplary case, genome-wide sequencing of a patient may reveal a variant in the EYA1 gene that has not been previously characterized and is of uncertain disease significance. If the genome-wide study was not performed because of an indication related to the EYA1 gene, a variant of uncertain significance (VUS) in this gene will not be reported to the clinician. However, the individual may have malformations of the kidneys, which are likely to be asymptomatic and will not be revealed by routine physical examination, but which place the patient at risk of chronic renal failure and the need for dialysis.

By applying reverse phenotyping as set out herein, a prompt is provided via the patient's electronic health record (EHR) that flags the possibility of renal malformations in the appropriately-consented patient. The judgment whether to proceed with further testing (e.g., abdominal ultrasound) is left to the clinician, and take into consideration their ability to explore whether the patient has other associated features (ear and craniofacial malformations, hearing loss, facial nerve paralysis) that can also be prompted by the reverse phenotyping system.

If the clinician finds further evidence supporting a pathogenic variant in EYA1 through physical examination or other testing (e.g., imaging, hearing), the clinician can then ask for a targeted re-analysis of the EYA1 gene by the clinical genetics diagnostic laboratory based on the results of the reverse phenotyping.

With this enhanced and focused phenotypic information, the laboratory can make a judgment about variant pathogenicity and issue a formal report, which will reveal the variant to the clinician for the first time. The provider is thus empowered to use their clinical judgement prompted by genomic sequence information, without having to be a specialist in clinical genetics or an expert in genomic medicine. They will instead be using their strengths in diagnostics in patient care, enhancing patient outcomes and associated costs.

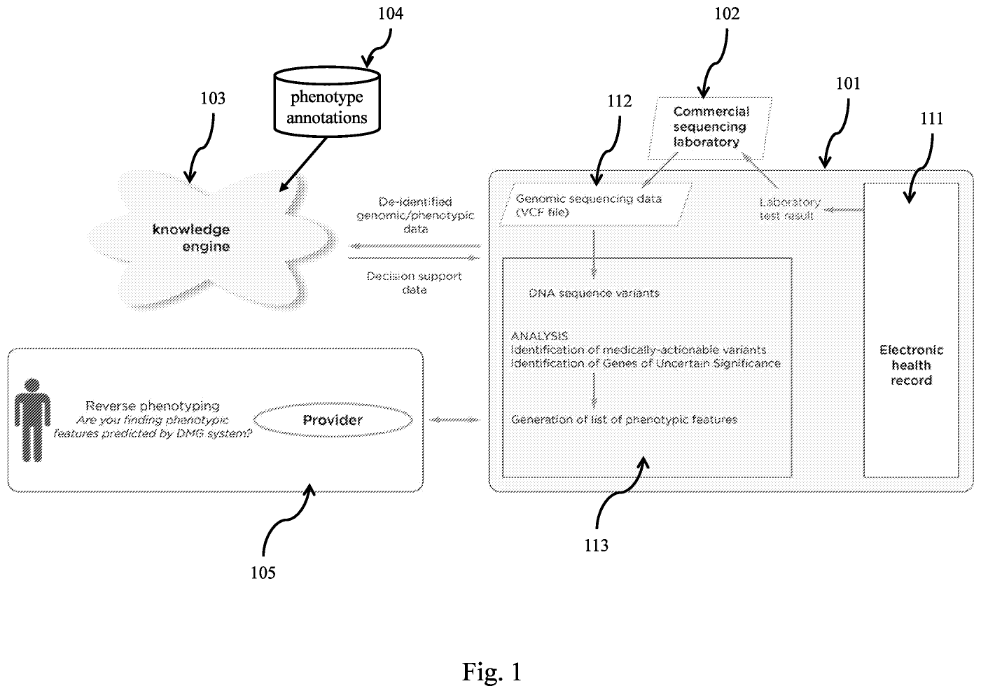

Referring now to FIG. 1, a schematic diagram is provided of a system for medical decision support employing reverse phenotyping according to embodiments of the present disclosure. Server system 101 accesses electronic health record system 111, which contain electronic health records for or more patients.

An electronic health record (EHR), or electronic medical record (EMR), may refer to the systematized collection of patient and population electronically-stored health information in a digital format. These records can be shared across different health care settings and may extend beyond the information available in a PACS. Records may be shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information.

EHR systems may be designed to store data and capture the state of a patient across time. In this way, the need to track down a patient's previous paper medical records is eliminated. In addition, an EHR system may assist in ensuring that data is accurate and legible. It may reduce risk of data replication as the data is centralized. Due to the digital information being searchable, EMRs may be more effective when extracting medical data for the examination of possible trends and long term changes in a patient. Population-based studies of medical records may also be facilitated by the widespread adoption of EHRs and EMRs.

Laboratory test results for a given patient are read from EHR system 111, and provided to a sequencing laboratory 102. Sequencing laboratory 102 provides genomic sequencing data 112, for example in the form of a Variant Call Format (VCF) file. It will be appreciated that the sequencing data for a given patient may be stored in the EHR for that patient. Accordingly, in some embodiments, a VCF file or equivalent may be read from the EHR instead of being obtained from a sequencing laboratory. Likewise, a VCF file may be cached for repeated use. It will also be appreciated that a variety of alternative file formats are available for storage and retrieval of genomic data of a patient, including Genomic VCF (gVCF), FASTA, FASTQ, SAM, GVF, and Genozip.

Variant calling is the process by which variants are identified from sequence data. IN an exemplary process, whole genome or whole exome sequencing is carried out to create FASTQ files. The sequences are aligned to a reference genome, creating BAM or CRAM files. Locations where the aligned reads differ from the reference genome are determined and written to VCF file.

As noted above, while whole genome sequencing is used in various embodiments, exome data or SNP data may be used in certain embodiments.

Subsystem 113 and knowledge engine 103 work in concert to extract the DNA sequence variants from the genomic sequencing data 112, identify medically-actionable variants, identify genes of uncertain significant (GUS), and generate a list of phenotypic features. In some embodiments, subsystem 113 is deployed within the same environment housing the electronic health record (EHR) system 111. The decision support data will be iteratively updated in the Knowledge Engine 103 as providers link genotype with phenotype across all sites using the system.

In some embodiments, subsystem 113 provides de-identified genomic/phenotypic data to knowledge engine 103, and receives base decision support data. In some embodiments, knowledge engine 103 is a cloud service, which may be deployed in a HIPAA compliant manner. It will be appreciated that a variety of methods may be used to integrate subsystem 113 with EHR system 111. For example, subsystem 113 may be provided as a plugin to EHR system 111. Subsystem 113 may also be resident on the same or another computer system as EHR system 111, and communicate with EHR system 111 via APIs exported by the EHR. Similarly, it will be appreciated that a variety of methods may be used to provide communication between EHR system 111 and knowledge engine 103. For example, knowledge engine 103 may export a RESTful API, a SOAP API, or APIs using any of various remote procedure call methods known in the art.

It will also be appreciated that in alternative embodiments, the functionality of the knowledge engine 103 and subsystem 113 may be combined in a single component that interacts with EHR system 111 using any of the aforementioned methods.

As pictured, a patient's genomic information is analyzed for variants. In some embodiments, variants are determined by subsystem 113 based on genomic sequencing data 112, while in some embodiments sequencing data 112 is provided to knowledge engine 103 for the detection of variants.

Variants may indicate certain diseases. Knowledge engine 103 has access to large and dynamic data sources, including external phenotype annotations 104. Exemplary sources of phenotypic data include the Human Phenotype Ontology (HPO), although it will be appreciated that a variety of other ontologies may be used, such as the Systematized Nomenclature of Medicine (SNOMED). In some embodiments, knowledge engine 103 determines additional phenotype annotations through artificial intelligence methods, discussed further below. Based on the available phenotype annotations, knowledge engine 103 returns phenotypic information to subsystem 113. In some embodiments, only medically actionable variants and/or genes of uncertain significance are considered when determining a list of phenotypic features.

The phenotypic features are provided by subsystem 113 to a review tool 105. In some embodiments, review tool 105 is a web-based application. In some embodiments, review tool 105 is an EHR interface. The phenotypic information is then reviewed by the clinician and after review, the clinician provides feedback. Physical review may include examining the patient for the indicated phenotypes, initiating additional testing, or other follow-up appropriate to the phenotype indicated. Identifying phenotypic features will allow targeted genetic testing and will prompt management of genetic conditions.

In various embodiments, subsystem 113 pulls data from and returns data to the EHR 111. For example, subsystem 113 can retrieve patient specific information to assist in determining a list of phenotypic features. Similarly, information regarding phenotypic features for assessment may be provided for storage in EHR 111. In some embodiments, subsystem 113 is configured to interface with application programming interfaces (APIs) of commercial sequencing laboratories.

Patient genomic data may be analyzed on a recurring schedule. For example, as phenotypic databases are updated, it may be desirable to prompt a provider to check for newly labeled phenotypes. Similarly, phenotypes which are not evident during a given examination may be flagged for re-evaluation at a future examination.

In some embodiments, the list of phenotypic features for evaluation is determined by reviewing the patient's variants against accessible databases of phenotype annotations (e.g., database 104).

In some embodiments, knowledge engine 103 comprises a learning system configured to determine phenotypic features for evaluation. For example, features provided to the learning system may include HPO terms and genotype, demographic information such as age, gender, or occupation, and comorbidity information. In such embodiments, the learning system is trained to provide an output list of phenotypic features for evaluation.

Similarly, in some embodiments, knowledge engine 103 comprises a learning system configured to determine new phenotype annotations based on variant and other patient information. In such embodiments, the learning system is trained based on observed phenotype information of a population of patients and their corresponding genomic information. In this way, otherwise undocumented links between variants and phenotypes can be inferred.

In some embodiments, a feature vector is provided to a learning system. Based on the input features, the learning system generates one or more outputs. In some embodiments, the output of the learning system is a feature vector.

In some embodiments, the learning system comprises a SVM. In other embodiments, the learning system comprises an artificial neural network. In some embodiments, the learning system is pre-trained using training data. In some embodiments training data is retrospective data. In some embodiments, the retrospective data is stored in a data store. In some embodiments, the learning system may be additionally trained through manual curation of previously generated outputs.

In some embodiments, the learning system, is a trained classifier. In some embodiments, the trained classifier is a random decision forest. However, it will be appreciated that a variety of other classifiers are suitable for use according to the present disclosure, including linear classifiers, support vector machines (SVM), or neural networks such as recurrent neural networks (RNN).

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

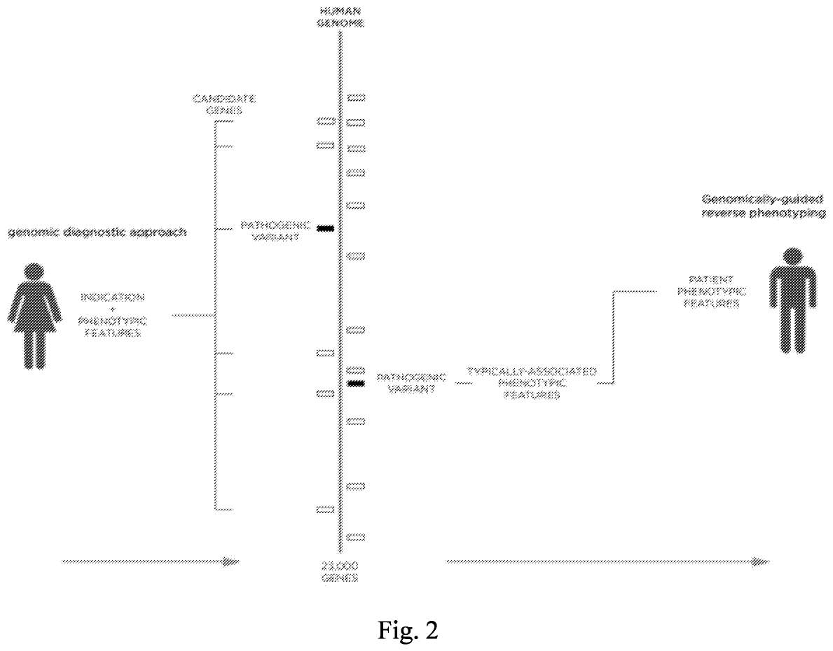

Referring now to FIG. 2, the reverse phenotyping approaches described herein are compared to an exemplary genomic diagnostic approach.

Genomic diagnostic approaches are illustrated on the left of FIG. 2. A patient presents with a disease or other indications, which cause a provider to initiate genetic testing. The phenotypic features of the presentation are used to identify a subset of the ˜23,000 genes in the human genome. If the sequence of one of these genes has a pathogenic variant, the disease and associated phenotypic features may be attributed to that variant.

Reverse phenotyping is illustrated on the right of FIG. 2. A gene with a clear pathogenic variant (or a variant of uncertain significance in a medically actionable gene) may be identified in genome-wide sequencing. When a gene is responsible for a disease, this relationship can be described by a set of phenotypic features, which are available in a variety of annotation databases. To ascertain whether the gene is causing a disease in the patient, the clinician can be prompted to look for these specific phenotypic features. This genetic sequence-prompted characterization of the patient is what is described as reverse phenotyping.

As set forth above, in various embodiments, a provider-facing application is provided within an EHR that is configured to prompt a clinician to examine a patient for specific phenotypic features. If some of the prompted phenotypic features are found in the patient, the clinician is advised to consider requesting a report about the specific gene (e.g., from a clinical genetics diagnostic laboratory), providing information about the phenotypic features identified in the patient. If the clinician's evaluation finds no evidence for phenotypic features associated with the genetic variant, this information is captured for further training of a learning system or dissemination to a public resource like ClinVar.

In some embodiments, variant exploration is provided for diagnostic laboratories. In some such embodiments, rather than integrating with an EHR, integration is provided with existing user interfaces for ordering or receiving the results of genomic testing from a clinical genetics diagnostic laboratory. For a patient who has given consent, the interaction with the diagnostic laboratory can thereby also include feedback about pathogenic variants and provide prompts for reverse phenotyping (as depicted, for example, on the right side of FIG. 2.

In various embodiments, the variant-phenotype association information that is collected from analysis of patient information is collected. As noted above, this information may be used to train learning systems to identify phenotypic information. It may also be provided to third parties for further use.

In various embodiments, deployment in a software as a service (SaaS) configuration is provided. Such a deployment may be remote from the health care organization or clinical diagnostic laboratory. In that case, genomic sequence and patient data are transferred to the remote system, which returns phenotypic prompts and associated information for patient management or diagnostic use.

It will be appreciated that the systems and method provided herein provide useful various features. Genomic information may be used to prompt the clinician to look for specific phenotypic features in a patient. The methods set out herein are applicable to genomic information pertaining to known pathogenic sequence variants in protein-coding sequences, and also to candidate variants in protein-coding sequences, variants in the remainder of the genome, structural and copy number variants, uniparental disomy, chimaerism, and epigenetic (transcriptional regulatory, DNA modifications, chromatin state, transcriptional state, cell subtype proportion changes) information. The genomic information can represent properties of the germ line genome as well as changes that have occurred in a subset of somatic cells. Reverse phenotyping can use current resources (ontologies such as the Human Phenotype Ontology (HPO), the Observational Medical Outcomes Partnership (OMOP)), or any others developed in the future, plus any other ontologies describing human phenotypes in terms of testing (such as and not restricted to imaging data, laboratory testing).

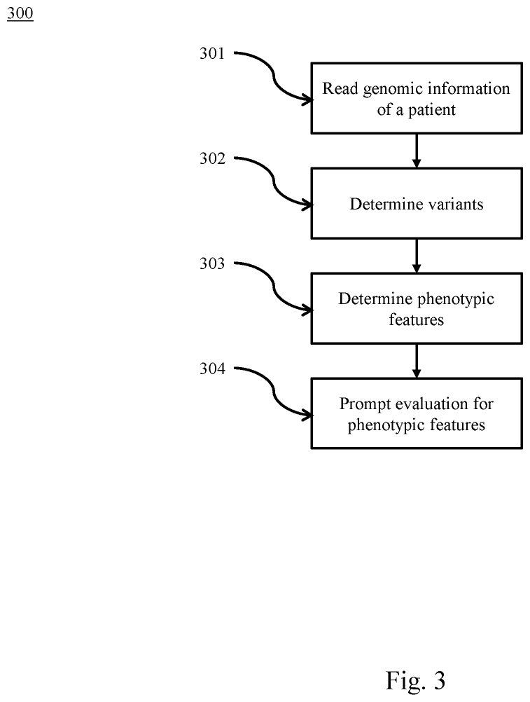

With reference to FIG. 3, a method for clinical decision support is illustrated according to embodiments of the present disclosure. At 301, genomic information of a patient is read from a datastore. At 302, one or more variant of the genomic information is determined. The one or more variant is associated with a disease state. At 303, one or more phenotypic features are determined, associated with the one or more variant. At 304, a healthcare provider is prompted to evaluate the patient for the one or more phenotypic features.

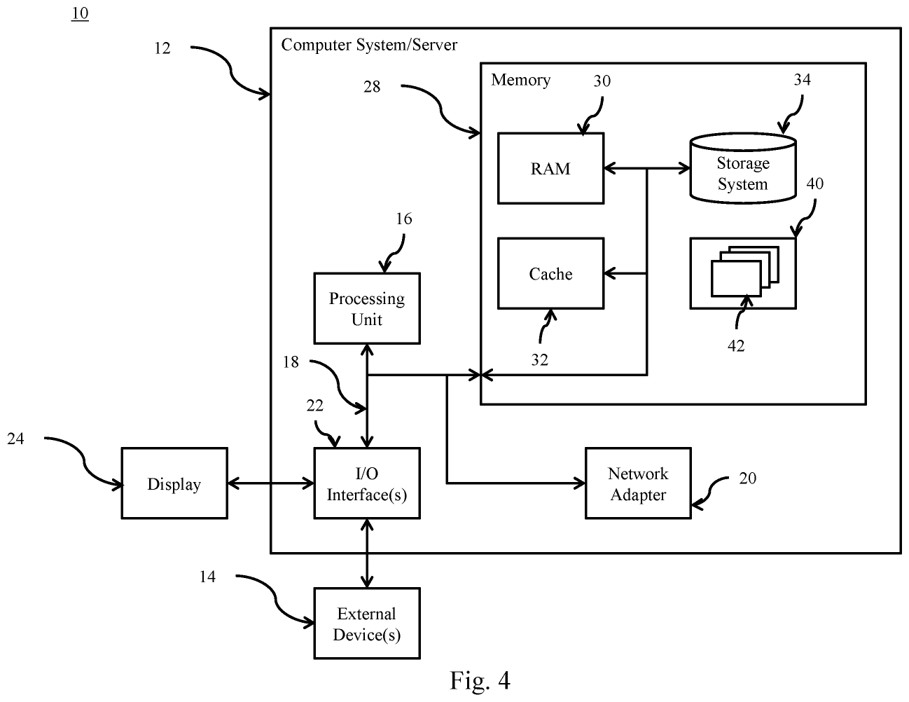

Referring now to FIG. 4, a schematic of an example of a computing node is shown. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 4, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, Peripheral Component Interconnect (PCI) bus, Peripheral Component Interconnect Express (PCIe), and Advanced Microcontroller Bus Architecture (AMBA).

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present disclosure may be embodied as a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A method comprising:
reading genomic information of a patient from a datastore;
determining one or more variant of the genomic information, the one or more variant being associated with a disease state;
determining one or more phenotypic features associated with the one or more variant;
prompting a healthcare provider to evaluate the patient for the one or more phenotypic features.

2. The method of claim 1, wherein reading the genomic information of the patient comprises accessing an electronic health record of the patient.

3. The method of claim 1, wherein reading the genomic information of the patient comprises accessing a sequencing provider.

4. The method of claim 1, wherein determining the one or more variant comprises comparing the genomic information of the patient to a reference sequence.

5. The method of claim 1, wherein determining the one or more variant comprises accessing a datastore containing associations between variants and disease states.

6. The method of claim 1, wherein determining the one or more phenotypic features comprises accessing a datastore containing associations between variants and phenotypes.

7. The method of claim 1, wherein determining the one or more phenotypic features comprises providing the one or more variant to a trained learning systems, and obtaining therefrom the one or more phenotypic features.

8. The method of claim 1, wherein prompting the healthcare provider comprises displaying the one or more phenotypic features in an electronic health record interface.

9. The method of claim 1, further comprising:
receiving from the healthcare provider an evaluation of the one or more phenotypic features in the patient.

10. The method of claim 9, further comprising:
providing the one or more variant and the evaluation of the one or more phenotypic features to a learning system, thereby training the learning system to associate the one or more variant and the one or more phenotypic features.

11. The method of claim 1, further comprising:
storing the phenotypic features of the patient in an electronic health record of the patient.

12. A system comprising:
a datastore containing genomic information of a patient;
a computing node comprising a computer readable storage medium having program instructions embodied therewith, the program instructions executable by a processor of the computing node to cause the processor to perform a method comprising:
reading the genomic information of the patient from the datastore;
determining one or more variant of the genomic information, the one or more variant being associated with a disease state;
determining one or more phenotypic features associated with the one or more variant;
prompting a healthcare provider to evaluate the patient for the one or more phenotypic features.

13. The system of claim 12, wherein reading the genomic information of the patient comprises accessing an electronic health record of the patient.

14. The system of claim 12, wherein reading the genomic information of the patient comprises accessing a sequencing provider.

15. The system of claim 12, wherein determining the one or more variant comprises comparing the genomic information of the patient to a reference sequence.

16. The system of claim 12, wherein determining the one or more variant comprises accessing a datastore containing associations between variants and disease states.

17. The system of claim 12, wherein determining the one or more phenotypic features comprises accessing a datastore containing associations between variants and phenotypes.

18. The system of claim 12, wherein determining the one or more phenotypic features comprises providing the one or more variant to a trained learning systems, and obtaining therefrom the one or more phenotypic features.

19. The system of claim 12, wherein prompting the healthcare provider comprises displaying the one or more phenotypic features in an electronic health record interface.

20-22. (canceled)

23. A computer program product for clinical decision support, the computer program product comprising a computer readable storage medium having program instructions embodied therewith, the program instructions executable by a processor to cause the processor to perform a method comprising:
reading genomic information of a patient from a datastore;
determining one or more variant of the genomic information, the one or more variant being associated with a disease state;
determining one or more phenotypic features associated with the one or more variant;
prompting a healthcare provider to evaluate the patient for the one or more phenotypic features.

24-33. (canceled)
